(19) United States
(12) Patent Application Publication
(10) Pub. No.: US 2010/0094055 A1
(43) Pub. Date: Apr. 15, 2010

(54) PROCESS FOR THE PREPARATION OF PHENETHYLAMINE DERIVATIVES

(75) Inventors: Rohit Ravikant Soni, Kalamaria (GR); Theocharis Koftis, Thessaloniki (GR); Theodoros Panagiotidis, Thessaloniki (GR); Ioanna Georgopoulou, Thessaloniki (GR)

Correspondence Address: AKC PATENTS, 215 GROVE ST., NEWTON, MA, 02466, US

(73) Assignee: PHARMATHEN S.A., Pallini-Attikis (GR)

(21) Appl. No.: 12/521,325

(22) PCT Filed: Jan. 9, 2007
(86) PCT No.: PCT/EP2007/000108

§ 371 Date: Jun. 26, 2009

(51) Int. Cl. C07C 211/03; C07C 209/00

(52) U.S. Cl. 564/375

(57) ABSTRACT

The present invention relates to an improved process for the preparation of essentially pure Venlafaxine Hydrochloride. Particularly, the process for the preparation of Venlafaxine Hydrochloride comprises the following steps: i) Preparation of 1-[Cyano-1-(4-methoxyphenyl)methyl]cyclohexanol, ii) Preparation of crude Venlafaxine Hydrochloride by reduction of 1-[Cyano-1-(4-methoxyphenyl)methyl]cyclohexanol with Alkali metal borohydride and Lewis acid and subsequent conversion to Venalfaxine hydrochloride with formic acid and paraformaldehyde and finally iii) Purification of crude Venlafaxine Hydrochloride.

### TECHNICAL FIELD OF THE INVENTION

The present invention relates to an improved process for the preparation of phenethylamine derivatives or salts or metabolites thereof and in particular for the preparation of essentially high pure Venlafaxine Hydrochloride or its metabolite ODV

### BACKGROUND OF THE INVENTION

Venlafaxine Hydrochloride, a structurally novel antidepressant for oral administration, is a synthetic phenethylamine bicyclic derivative, chemically unrelated to tricyclic, tetracyclic, or other available types of antidepressant agents. It is usually categorized as a serotonin-norepinephrine reuptake inhibitor (SNRI), but it has also been referred to as a serotonin-norepinephrine-dopamine reuptake inhibitor.

Venlafaxine Hydrochloride is chemically designated as (R/S)-1-[2-(dimethylamino)-1-(4-methoxyphenyl)ethyl]cyclohexanol hydrochloride, or (±)-1-[a[α-(dimethylamino)methyl]p-methoxybenzyl]cyclohexanol hydrochloride and it has the empirical formula of C₁₇H₂₇NO₂.HCl.

Venlafaxine Hydrochloride presents the following structural formula I:

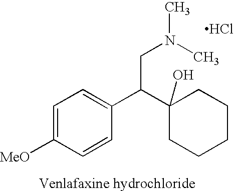

Venlafaxine is used in the form of its hydrochloride salt, which is more desirable since Venlafaxine can be more efficiently formulated. This is important, because formulations need to meet certain pharmaceutical requirements and specifications. Venlafaxine, as its hydrochloride salt, can be easily formulated in the form of tablets, capsules, lozenges, powders, and other forms for oral administration.

Prior art processes for the preparation of Venlafaxine do not provide the desired yields during the chemical reactions. Furthermore, the compound often comprises significant amounts of unwanted by-products and the reaction may require a long period of time to be completed.

Moreover, prior art processes also present the disadvantage of non-satisfactory purity and yield of the product.

EP-A-112 669 and its corresponding U.S. Pat. No. 4,535,186 discloses a process for the preparation of Venlafaxine by methylating 1-[2-Amino-1-(4-methoxyphenyl)ethyl]cyclohexanol with a mixture of formaldehyde and formic acid in water to form the Venlafaxine base, wherein said 1-[2-Amino-1-(4-methoxyphenyl)ethyl]cyclohexanol is being prepared by reacting 4-methoxyphenyl acetonitrile with Cyclohexanone in the presence of n-butyl lithium to form 1-[Cyano-1-(4-methoxyphenyl)methyl]cyclohexanol.

This document discloses various reduction conditions as under i) Pd/C and hydrogen in ethanol+THF media, ii) Lithium aluminium hydride in acid media, iii) Rhodium Alumina in ammoniacal ethanol, iv) Borane tetrahydrofyran complex.

However the process of the above patent has the disadvantage that the addition of n-butyl lithium to 4-methoxyphenyl acetonitrile is hazardous, requiring high safety measures and great attention in handling butyl lithium, in order to avoid any unwanted incidents during the preparation process.

WO-A-03/050074 discloses an alternative method by reduction 1-[Cyano-1-(4-methoxyphenyl)methyl]cyclohexanol using Raney nickel without pretreatment in methanolic ammonia.

Although each of the above patents represents an attempt to overcome the use of costly hazardous reducing agent or organic catalyst, there still exists a need for a cheaper and safer process which provides a higher yield with higher purity.

### SUMMARY OF THE INVENTION

It is, therefore, an object of the present invention to provide an improved process for the preparation of essentially pure Venlafaxine Hydrochloride or its metabolite ODV, which overcomes the deficiencies of the prior art and results to an increased purity and yield of Venlafaxine Hydrochloride.

Another object of the present invention is to provide an improved method of preparing Venlafaxine Hydrochlorideor its metabolite ODV, by minimizing the presence of any contaminants and formed by-products during the reactions, thus increasing the purity level of Venlafaxine Hydrochloride.

Another object of the present invention is to provide an improved method of preparing Venlafaxine Hydrochloride or its metabolite ODV by selecting the appropriate reactants, solvents and catalysts used during the organic reactions, so that the purity and yield of reaction are increased.

Further object of the present invention is to provide a method of preparing Venlafaxine Hydrochloride or its metabolite ODV, which results to reduction of the cost of production.

In accordance with the above objects of the present invention, a process for the preparation of Venlafaxine Hydrochloride or its metabolite ODV is provided comprising

a) reduction of 1-[Cyano-1-(4-methoxyphenyl)methyl]cyclohexanol, with alkali metal borohydride and Lewis acid to get 1-[2-Amino-1-(4-methoxyphenyl)ethyl]cyclohexanol and

b) further conversion of the 1-[2-Amino-1-(4-methoxyphenyl)ethyl]cyclohexanol to Venlafaxine hydrochloride or its metabolite ODV.

Preferred embodiments of the present invention are set out in dependent claims 2 to 7.

According to the present invention, Venlafaxine Hydrochloride or its metabolite ODV is prepared from 1-[Cyano-1-(4-methoxyphenyl)methyl]cyclohexanol, which is used as the key starting material.

1-[Cyano-1-(4-methoxyphenyl)methyl]cyclohexanol is reduced with Alkali metal borohydride such as LiBH₄,NaBH₄, KBH₄ and cheaper Lewis acid such as AlCl₃, ZnCl₂, SnCl₂ giving 1-[2-amino-1-(4-methoxyphenyl)ethyl]cyclohexanol.

This compound without isolation in its salt form is further converted to Venlafaxine hydrochloride by reacting with formic acid and paraformaldehyde and subsequent reaction with isopropanolic hydrogen chloride.

The isolated crude Venlafaxine hydrochloride has purity >99.0%, which is further purified by single crystallization in refluxing isopropanol to >99.8% purity.

The high purity of the crude form is attributed because of the fine adjustment in the molar ratio of the alkali metal borohydride and Lewis acid so that after quenching the reaction mass in water it does not generate any extreme acidic or basic conditions which lead to undesired impurities.

The present invention provides a process where corresponding phenylacetonitrile derivatives are reduced chemically using alkali metal borohydride along with the most inexpensive Lewis acid, which overcomes the disadvantage associated with heterogeneous catalytic reduction. The molar proportion of the alkali metal borohydride and Lewis acid is so well optimized that it results in highly pure phenylethylamine derivatives which on subsequent reaction with formic acid and paraformaldehyde provide Venlafaxine hydrochloride with higher purity.

### DETAILED DESCRIPTION OF THE INVENTION

The present invention relates to an improved process for the manufacture of the compound 1-[2-amino-1-(4-methoxyphenyl)ethyl]cyclohexanol and methylation of said compound to produce the compound 1-[2-dimethyl(p-methoxyphenyl)ethyl]cyclohexanol or venlafaxine

According to the present invention, the process for the preparation essentially pure Venlafaxine Hydrochloride or its metabolite ODV comprises the following steps:

### Preparation of 1-[Cyano-1-[(4-methoxyphenyl)methyl]cyclohexanol

This intermediate is of significant importance for use in the preparation of Venlafaxine Hydrochloride; therefore it is selected as the key starting material for the present process.

This intermediate is prepared according to the process disclosed in Chinese Patent CN 1,225,356.

The preparation of 1-[Cyano-1-[(4-methoxyphenyl)methyl]cyclohexanol involves the reaction of 4-methoxyphenylacetonitrile with Cyclohexanone in the presence of a solvent mixture comprising a solution of basic material, such as alkali metal alkoxide, alkali amide or alkali hydride and alcohol. The solid mass obtained by the reaction is then filtered to prepare 1-[Cyano-1-[(4-methoxyphenyl)methyl]cyclohexanol, which constitutes the starting material for the preparation of Venlafaxine Hydrochloride.

The alcohol used for the preparation of 1-[Cyano-1-[(4-methoxyphenyl)methyl]cyclohexanol may be selected from alcohols, such as methanol, ethanol, isopropanol, n-propanol, ethyleneglycol, glycerol, propanediol, butanediol, butanetriol and others.

The concentration of the alcohol plays an important role in preparing 1-[Cyano-1-[(4-methoxyphenyl)methyl]cyclohexanol, as it determines the reaction rate. The concentration of the alcohol is preferably between 1% and 30% by weight, and more preferably between 5% and 20% by weight. If the concentration of the alcohol is very low, then the rate of the reaction rate is very slow. If the concentration of the alcohol is very high, then the reaction is realized very quickly, but a significant amount of unwanted by-products is produced during the side reactions.

Sodium methoxide, which is an alkoxide, works as a strong base and is used as an intermediary in the reaction of 4-Methoxyphenylacetonitrile with Cyclohexanone. It acts by abstracting proton from 4-Methoxyphenylacetonitrile to generate carbanion, which reacts with cyclohexanone to provide the 1-[Cyano-1-[(4-methoxyphenyl)methyl]cyclohexanol after quenching in water.

The solid mass obtained by the reaction between 4-Methoxyphenylacetonitrile and Cyclohexanone is then filtered by the use of solvent toluene and water to prepare 1-[Cyano-1-[(4-methoxyphenyl)methyl]cyclohexanol. During the process, water is used as the bulk solvent, whereas toluene is used as the co-solvent.

The above mentioned reaction is conducted at 45-50° C. The reaction temperature is also very significant parameter, as it can determine the reaction rate. A low temperature results to a slow reaction rate, whereas a high temperature results to many side-reactions, thus increasing the amount of impurities produced.

The preparation of 1-[Cyano-1-[(4-methoxyphenyl)methyl]cyclohexanol is described in the following synthetic scheme:

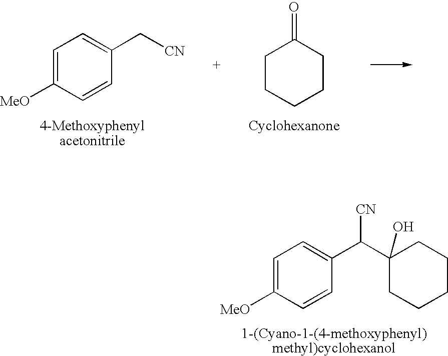

In condensation NaOCH₃,NaOC₂H₅,NaNH₂, and NaH are used as a base

### Step I: Preparation of Venlafaxine Hydrochloride-Crude

The preparation of Venlafaxine Hydrochloride (crude) comprises the reduction of 1-[Cyano-1-(4-methoxyphenyl)methyl]cyclohexanol with Alkali metal borohydride and Lewis acid producing 1-[2-Amino-1-(4-methoxyphenyl)ethyl]cyclohexanol.

The intermediate 1-[2-amino-1-(4-methoxyphenyl)ethyl]cyclohexanol formed is not isolated; it is further reacted with formic acid and paraformaldehyde followed by reaction with isopropanolic HCl, to yield crude Venlafaxine Hydrochloride or its metabolite ODV.

The synthetic scheme for the preparation of Venlafaxine Hydrochloride or its metabolite ODV as crude is as follows:

### Step I: Preparation of 1-[2-Dimethylamino-1-(4-methoxyphenyl)ethyl]cyclohexanol

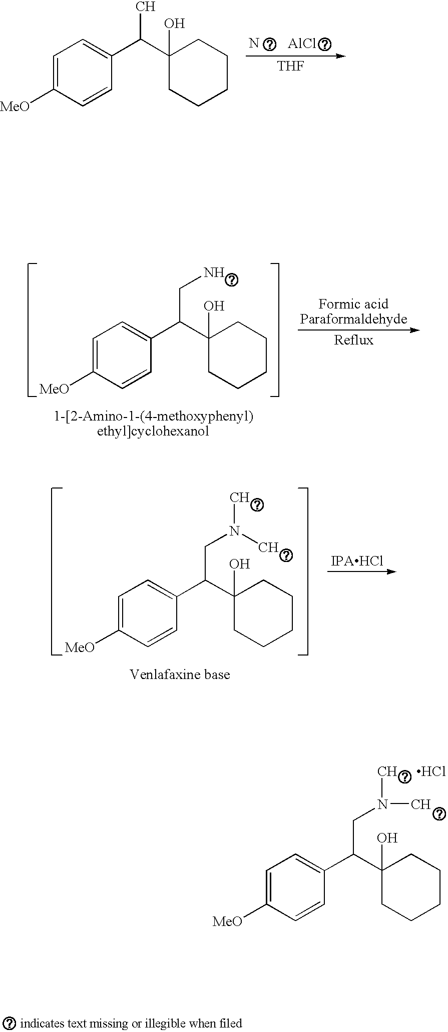

In Step I, 1-[Cyano-1-(4-methoxyphenyl)methyl]cyclohexanol (I) is reduced by alkali metal borohydride and Lewis acid, such as AlCl₃, ZnCl₂, SnCl₂, forming 1-[2-Amino-1-(4-methoxyphenyl)ethyl]cyclohexanol (II).

The reaction can be carried out in ethereal solvent, such as tetrahydrofuran, 2-Methyl tetrahydrofuran, Diethyl ether, Diisopropyl ether, Methyl-t-butyl ether, 1,2-Dimethoxy ethane, 1,4-Dioxane and most preferably tetrahydrofuran.

The reduction is carried out at a temperature of between 15 to 45° C., and most preferably at 35-40° C. for preferably 12 to 30 hours and most preferably 20 to 24 hours.

The reaction is performed in an inert atmosphere of Nitrogen or Argon. The molar ratio of (I), MBH₄ and Lewis acid is preferably kept at 1:2.5-4.5:4.0-6.0 (wherein M can be Li, Na and K) and most preferably 1:3.5:5.0. The progress of the reaction is monitored both on TLC and HPLC.

After completion of the reaction, excess THF is removed by distillation and the reaction mass is quenched with alkali solution and extracted with a water immiscible solvent like toluene or ethyl acetate. The organic layer is concentrated to obtain phenylethylamine derivative as oily residue which is then treated with paraformaldehyde and formic acid for alkylation as per the prior art.

After complete conversion of the amino and Spiro compound to Venlafaxine, the reaction mass is acidified and extracted with ethyl acetate to remove impurities and then the aqueous layer is basified to pH>12 and extracted with ethyl acetate. The combined organic layer is acidified to pH<2.0 with isopropanolic HCl to yield Venlafaxine hydrochloride crude after filtration and drying.

During the whole process of the present invention 1-[2-Amino-1-(4-methoxyphenyl)ethyl]cyclohexanol and Venlafaxine base are not isolated and taken directly to the next step.

The presence of the salt of formic acid allows the methylation of the precursor 1-[2-Amino-1-(4-methoxyphenyl)ethyl]cyclohexanol to proceed very quickly with high yield, minimizing the formation of undesirable by-products.

During the process of the present invention, water is used as a bulk solvent and toluene as a co-solvent to remove any non-basic impurities. The use of water as a bulk solvent presents the advantage that the cost of the manufacture of Venlafaxine Hydrochloride is reduced giving good yield and purity.

Ethyl acetate is a non-polar solvent and it is used as an anti-solvent used to extract the soluble reaction mass.

### Step II: Purification and Crystallization of Crude Venlafaxine hydrochloride

The second step of the preparation process of the present invention is the purification of Crude Venlafaxine Hydrochloride in refluxing Isopropanol. This step is disclosed in EP-B-112 669 for the preparation of Venlafaxine Hydrochloride, having the polymorphic form C.

The purification process can be depicted in the following scheme:

### Step II: Purification of Venlafaxine Hydrochloride-Crude

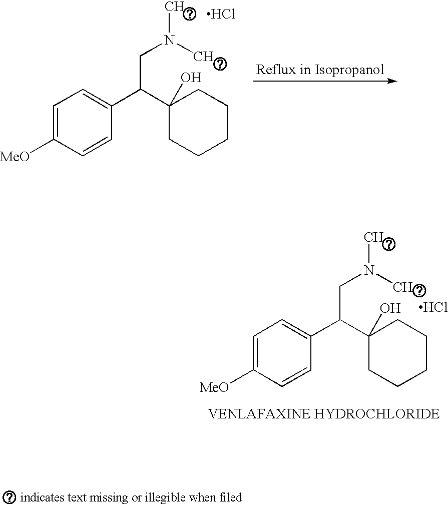

The crude form of Venlafaxine Hydrochloride produced in Step I is then refluxed in Isopropanol at a temperature of 25-30° C. After filtration, washing of the wet solid mass with isopropanol and drying of the white precipitate under vacuum at 45-50° C., the loss on drying is less than 0.5%.

The process of the present invention will be demonstrated in more details with reference to the following examples, which are provided by way of illustration only and should not be construed as limit to the scope of the reaction in any manner.

### Example 1

### Preparation of 1-[Cyano-1-[(4-methoxyphenyl)methyl]cyclohexanol

The following raw materials were used for the preparation of the intermediate:

TABLE 1
Raw materials

| | | |
|---|---|---|
| 4-Methoxyphenylacetonitrile | 10 | g (0.068 mol) |
| Cyclohexanone | 8.75 | g (0.089 mol) |
| Sodium methoxide in methanol (25% w/v) | 47 | ml (0.203 mol) |
| Toluene | 100 | ml |
| D.M. Water | 110 | ml |

The intermediate was prepared according to the following process:

Sodium methoxide solution in methanol (47 ml, 25% w/v) was formed into a glass assembly under Argon atmosphere. The solution was cooled under stirring to −5° C., and 4-Methoxyphenyl acetonitrile (10 g, 0.068 mol) was added slowly at −3 to −5° C. The reaction mixture was maintained at −3 to −5° C. for 2 hours under stirring. Then cyclohexanone (8.75 g, 0.089 mol) was added to the reaction mass and the resulting reaction mixture was maintained at temperature between −3 and −5° C. for 10 to 12 hours under stirring till completion of the reaction on TLC. Water (100 ml) was added slowly to the reaction mass during the period of 30 minutes while maintaining the temperature between 0 to 2° C. Subsequently, the reaction mass was filtered and washed with water (10 ml), and the wet cake was well sucked dry. Then, the wet solid was transferred to another assembly containing toluene (100 ml), and the mixture was heated slowly to about 50° C. under stirring till a clear solution. The hot clear solution was then filtered, cooled slowly to 5-10° C., maintained for 30 minutes and filtered through the Buchner funnel. The wet cake was then well sucked dry and dried at about 45-50° C. under vacuum till loss of drying was less than 0.5%. The yield of the purified product was 13.3 g (80%).

### Example 2

### Preparation of Venlafaxine Hydrochloride or its Metabolite ODV-Crude

The following raw materials were used for the preparation of 1-[2-Dimethylamino-1-(4-methoxyphenyl)ethyl]cyclohexanol:

TABLE 2
Raw materials

| | | |
|---|---|---|
| 1-[Cyano-1-[(4-methoxyphenyl) methyl] | 25.0 | g (0.102 moles) |
| cyclohexanol (KSM) | | |
| Sodium borohydride (NaBH₄) | 13.5 | g. (0.357 moles) |
| Aluminum chloride (AlCl₃) | 68.0 | g (0.51 moles) |
| Tetrahydrofuran (THF) | 350 | ml |
| Toluene | 375 | ml |
| Ethyl acetate | 175 | ml |
| Aq. Hydrochloric acid (~1%) | 350 | ml |
| 50% Sodium hydroxide solution | 180 | ml |
| Formic acid | 16.5 | ml |
| Paraformaldehyde | 4.5 | ml |
| Isopropanolic HCl (8-10%) | 20 | ml |
| D.M. Water | 100 | ml |

Tetrahydrofuran THF (250 ml) was charged in a 3 neck one liter R.B.flask equipped with overhead stirrer, addition funnel and reflux condenser in an inert atmosphere of argon at 25-30° C. and AlCl₃ (68.0 g., 0.51 moles) was slowly added in small lots maintaining temperature between 25 to 30° C. for 30 minutes. To this light yellow colored solution of AlCl₃ in THF, Sodium borohydride (13.5 g, 0.357 mole) and 1-[Cyano-1-(4-methoxyphenyl)methyl]cyclohexanol (KSM) (25.0 g., 0.102 moles) were slowly added in small portions while maintaining the temperature between 25 to 30° C. After 5 to 10 minutes, the resulting suspension was heated to about 40 to 50° C. and maintained under stirring till completion for 20-24 hours. (Reaction monitoring on TLC at this stage indicated the completion of reaction). The reaction mass was cooled to 25-30° C. and quenched with 50% sodium hydroxide solution (150 ml) very slowly over a period of 60-90 minutes maintaining the temperature at 25-30° C., and extracted out with toluene (3×50 ml) to remove non basic impurities. Combined organic layer was concentrated in rotavapor under reduced pressure to yield amino base as viscous oil.

In another assembly, the resultant amino base, water (200 ml), paraformaldehyde (9.0 g) and formic acid (27.5 ml) were mixed under stirring. The reaction mass was heated under reflux at 95-100° C. till completion of reaction on HPLC/TLC for 24-28 hours. The reaction mass was cooled to 25-30° C. and extracted with ethyl acetate (2×25 ml). The separated organic layer was discarded and the aqueous layer was basified with 50% aqueous sodium hydroxide solution to pH>12 and extracted with ethyl acetate (2×50 ml). The layers were separated, the organic layers were combined, dried on a anhydrous sodium sulphate and transferred to another assembly and acidified with ˜20% isopropanolic HCl till pH<2.0 under vigorous stirring at 25-30° C. The reaction mass was maintained under stirring at 25-30° C. for another 30 minutes, the separated solid was filtered and the wet cake was washed with isopropanol (25 ml), and dried at 45-50° C. under vacuum to yield Crude Venlafaxine hydrochloride (15.0-17.0g, 47-53% overall yield).

### Example 3

### Preparation of Venlafaxine Hydrochloride-Crude

Tetrahydrofuran (250 ml) is charged to a suitable 3 neck R.B.flask equipped with overhead stirrer, addition funnel and reflux condenser in an inert atmosphere of argon at 25-30° C. and zinc chloride (69.5 g., 0.51 moles) was slowly added in small lots maintaining temperature between 25 to 30° C. for 30 minutes. To this solution of ZnCl₂ in THF, Sodium borohydride (13.5 g, 0.357 mole) and 1-[Cyano-1-(4-methoxy phenyl)methyl]cyclohexanol (25.0 g.,0.102 moles) are added in small portions maintaining the temperature between 25 to 30° C. Reaction mass is then further heated to 45-50° C. and maintained under stirring till completion of the reaction (20-24 hours). Work up and further reaction to formic acid and paraformaldehyde was performed according to the process of Example 2 and provided Venlafaxine.HCl-Crude in similar yield and quality.

### Example 4

### Preparation of Venlafaxine Hydrochloride-Crude

Tetrahydrofuran (250 ml) was charged to a suitable 3 neck R.B.flask equipped with overhead stirrer, addition funnel and reflux condenser in an inert atmosphere of argon at 25-30° C. and stannous chloride (115.0, 0.51 moles) was slowly added in small lots maintaining the temperature between 25 to 30° C. for 30 minutes. To this solution of SnCl₂ in THF, Sodium borohydride (13.5 g, 0.357 mole) and 1-[Cyano-1-(4-methoxy phenyl)methyl]cyclohexanol (25.0 g., 0.102 moles) were added in small portions maintaining the temperature between 25 to 30° C. Reaction mass was then further heated to 45-50° C. and maintained under stirring till completion of the reaction (20-24 hours). Work lip and further reaction to formic acid and paraformaldehyde was performed according to the process of Example 2 and provided Venlafaxine.HCl-Crude in similar yield and quality.

### Example 5

### Purification of Venlafaxine Hydrocloride-Crude

Isopropanol (90 ml) and Venlafaxine Hydrochloride (15.0 g.) were charged in a 3 neck 250 ml one liter R.B.flask equipped with an overhead stirrer, an addition funnel and a reflux condenser at 25-30° C. The resultant suspension was heated to reflux (˜80° C.). The reflux was maintained for 30-60 minutes and filtered hot through the celite bed and washed with hot isopropanol (5 ml). The filtrate was allowed to cool to 25-30° C. under stirring, and then it was further cooled to 10-15° C. and filtered through the Buchner funnel. The wet cake of pure Venlafaxine hydrochloride was washed with cold Isopropanol (5 ml) and then suck dried for a period of 15-30 minutes. The white precipitate was then dried under vacuum at 45-50° C. till loss on drying was lee than 0.5%. The dried Venlafaxine Hydrochloride provided had a purity of >99.8% (13.0-13.5 g, 86-90%).

Therefore, the present invention describes a method of preparing essentially pure Venlafaxine Hydrochloride in an improved manner This method comprises the preparation of Venlafaxine Hydrochloride in a crude form (Step I) and its purification and crystallization processes (Step II).

While the present invention has been described with respect to the particular embodiments, it will be apparent to those skilled in the art that various changes and modifications may be made in the invention without departing from the spirit and scope thereof, as defined in the appended claims.

1. A process for the preparation of high purity Venlafaxine Hydrochloride or its metabolite ODV, which comprises:
a) reduction of 1-[Cyano-1-(4-methoxyphenyl)methyl]cyclohexanol, with alkali metal borohydride and Lewis acid to get 1-[2-Amino-1-(4-methoxyphenyl)ethyl]cyclohexanol and
b) further conversion of the 1-[2-Amino-1-(4-methoxyphenyl)ethyl]cyclohexanol to Venlafaxine hydrochloride or its metabolite ODV.

2. The process according to claim 1, wherein the reduction of 1-[1-Cyano-1-(4-methoxy phenyl)methyl]cyclohexanol is carried out with alkali metal borohydride such as LiBH₄,NaBH₄, KBH₄ and Lewis acid such as AlCl₃, ZnCl₂ and SnCl₂.

3. The process according to claim 2, wherein the molar ratio of 1-[1-Cyano-1-(4-methoxyphenyl)methyl]cyclohexanol:alkali metal borohydride:Lewis acid is preferably between 1:2.5-4.5:4.0-6.0, and more preferably between 1:3.5-4.0:4.5-5.5.

4. The process according to claim 1, wherein said conversion is carried out by reaction of the 1-[2-Amino-1-(4-methoxyphenyl)ethyl]cyclohexanol with formic acid and paraformaldehyde in excess of water.

5. The process according to claim 4, wherein after forming the compound of Venlafaxine hydrochloride or its metabolite, the product is purified by reaction with isopropanol.

6. A process for the preparation of 1-[2-Amino-1-(4-methoxyphenyl)ethyl]cyclohexanol and further its conversion to Venlafaxine hydrochloride or its metabolite without isolation of its freebase

7. Venlafaxine hydrochloride of high purity or its metabolite ODV prepared according to the process as defined in claim 1.
